(19) United States
(12) Patent Application Publication
(10) Pub. No.: US 2011/0287550 A1
(43) Pub. Date: Nov. 24, 2011

(54) Method for continuously monitoring solution-phase synthesis of oligonucleotide

(75) Inventors: Jentaie Shiea, Kaohsiung (TW); Chu-Nian Cheng, Kaohsiung (TW)

(73) Assignees: National Sun Yat-Sen University, Kaohsiung (TW); ScinoPharm Taiwan, Ltd., Tainan County (TW)

(21) Appl. No.: 13/114,516

(22) Filed: May 24, 2011

Related U.S. Application Data
(60) Provisional application No. 61/347,511, filed on May 24, 2010.

(51) Int. Cl. G01N 33/52; G01N 21/00

(52) U.S. Cl. 436/94; 422/82.05

(57) ABSTRACT

The present invention provides a system and method for real-time continuously monitoring of oligonucleotide synthesis in solution phase.

### CROSS REFERENCE TO RELATED APPLICATIONS

The present application claims priority to Provisional Patent Application 61/347,511, filed May 24, 2010, the contents of which is here incorporated by reference.

### BACKGROUND OF THE INVENTION

### Summary of the Invention

The present invention relates to a system under atmospheric pressure and low-moisture environment for real-time continuously monitoring oligonucleotides synthesis in solution phase, wherein the said system combines electrospray-assisted laser desorption ionization (ELDI) with mass spectrometer and a reactor. The ionization source part of ELDI and the reactor containing carbon powders and the reaction mixture of oligonucleotide synthesis are isolated in a nitrogen-filled chamber. While the reactor is charged with nitrogen gas, a trace of the solution in the reactor is pushed into a capillary. The sample solution flowed out of the capillary is desorbed by laser, and then the desorbed gaseous analyte molecules, including neutral oligonucleotides, are ionized by a ESI device to generate ESI-like analyte ions. The produced analyte ions are detected by the mass spectrometer connected with the liquid-ELDI device.

The present invention generally allows for the analysis of air and moisture sensitive reactions in a continuous manner.

In an embodiment of the present invention, a liquid electrospray-assisted laser desorption/ionization (liquid-ELDI) combined with an ion trap mass spectrometer was used to continuously and simultaneously monitor the synthesis of RNA tetramers, coupling RNA trimers with RNA monomers (3+1 mer). Since RNA synthesis is rather sensitive to moisture, the monitoring must be carried out under anhydrous condition. If the monitoring of the RNA synthesis is carried out under ambient conditions, the undesired oxidized byproduct will be formed, and then the analyte ion signals of the original products cannot be detected. To effectively lower the humidity during the measurement by isolating the ionization device and the reactor in a closed chamber, e.g. reducing the moisture content in the reactor and the connected pipelines, is critical to the success of continuous monitoring of RNA synthesis. The reactor containing RNA trimers and RNA monomers was filled with nitrogen gas to remove moisture. While the reactor was charged with more nitrogen, a trace of the solution in the reactor was pushed into a capillary. The sample solution flowed out of the capillary was desorbed by laser, and then the desorbed gaseous analyte molecules, such as neutral RNA monomers, trimers or tetramers, were ionized by ESI device to generate ESI-like analyte ions. The produced analyte ions were detected by the ion trap mass spectrometer connected with the liquid-ELDI device.

In one embodiment of the present invention, the real-time and continuous monitoring for the synthesis of RNA tetramers (3+1 mer) was successfully achieved. The results indicate that the use of carbon powders and the ESI solution while conducting liquid-ELDI does not interfere with the detection of reactants and products.

### THE ADVANTAGE OR CHARACTERISTICS OF THIS INVENTION

Products of RNA synthesis exhibit good stability in atmospheric-pressure environment. However, the synthetic reaction must be carried out in highly anhydrous conditions at all times. Otherwise, trace water from the moisture would react with the reactants to form an oxidized byproduct. Therefore, it is necessary to design an analytical system that can provide a low-humidity environment to perform real-time and continuous monitoring for the RNA synthesis, which can successfully monitor the change of the compositions in the reaction solution. The system of the present invention can achieve the aforementioned objectives and aid the understanding of the mechanism and kinetics of the reaction. Accordingly, the system could facilitate process improvements, increase the yield, and be further applied in quality management during plant production.

Liquid-ELDI allows analyte ions to be generated directly from organic solvents or aqueous solutions of the solution sample. So, the analyte ion signals can be successfully monitored by the system of the present invention. Therefore, the present invention provides an analytical technique for continuous monitoring the states of ongoing chemical reactions occurring in various solvents.

Since oligonucleotide synthesis is rather sensitive to moisture, its reaction monitoring must be undertaken under anhydrous condition. If the continuous or non-continuous monitoring of the oligonucleotide synthesis is carried out under ambient conditions, nucleotide blocks would be exposed to moisture of the atmosphere and then converted to an oxidized byproduct. Consequently, the analyte ion signals of the original products cannot be detected. Therefore, to continuously monitoring a chemical reaction sensitive to moisture, it is necessary to develop a real-time system which contains liquid-ELDI, mass spectrometer and a reactor under controlled environmental conditions. In one embodiment of the present invention, the system that contains the nitrogen-filled chamber with a reactor inside, liquid-ELDI and ion trap mass spectrometer can respond rapidly to the change of the chemicals (including reactants, intermediates, and products) present in the reaction mixture of the oligonucleotide synthesis.

### BRIEF DESCRIPTION OF THE DRAWINGS

FIG. 1 shows the monitoring system of one embodiment of the present invention, containing an IR laser device, an ESI device, an ion trap mass spectrometer and a reactor, wherein the reactor and the ESI device are in a closed chamber. The insert at the lower left corner of FIG. 1 is an enlargement of the said closed chamber.

FIG. 2 shows the schematic diagrams of outside of the closed chamber of FIG. 1. FIG. 2(A) shows the front and right side views from the end connected to the mass spectrometer. FIG. 2(B) shows the front and left side views from the end connected to the mass spectrometer.

FIG. 3 shows the schematic diagram of external pipelines on the right side of the closed chamber as shown in FIG. 2(A).

FIG. 4 shows the enlarged layout of internal pipelines connected to the reactor within the closed chamber. Pipelines (1) to (3) are the solution inlet 1, the nitrogen inlet 1 and the solution inlet 2 as shown in FIG. 3, having a 0.55 mm inner diameter. Pipeline (4) is a sampling outlet, having a 10 mm inner diameter. Pipeline (5) is a gas outlet, which is used to release the inner pressure of the reactor during injecting the solutions containing reactants into the reactor, having a 0.8 mm inner diameter.

FIG. 5 shows the insides of the closed chamber, including the reactor, the ESI device, and a humidity and temperature meter. FIG. 5(A) shows a photo of the closed chamber. FIG. 5(B) shows a schematic diagram of the closed chamber.

FIG. 6 show the extracted ion chromatograms of analytes obtained from the 3+1 mer oligonucleotide synthesis by using one embodiment of the monitoring system of the present invention: (a) m/z 861 (1 mer), (b) m/z 1282 (3 mer), (c) m/z 1738 (4 mer-DMT), and (d) m/z 2041 (4 mer).

FIG. 7 shows the average mass spectrograms obtained during different intervals of the 3+1 mer oligonucleotide synthesis by using one embodiment of the monitoring system of the present invention: (a) 2.1-8.9 min; (b) 21.7-30.4 min; (c) 31.3-37.8; and (d) 40.7-55.6 min.

FIG. 8 describes the hexamer (5+1 mer) RNA synthesis.

FIG. 9 shows the reaction scheme of 5+1 mer RNA synthesis.

FIG. 10 shows the reaction mechanism of dead 1 mer, produced from water and activated 1 mer, which dead 1mer is in turn reacted with another activated 1mer to form a dimer.

FIG. 11 shows a correlation chart between the amount of the acid and the ratio of the width of the peak of the monovalent ion signal of 5 mer or 6 mer to dimer+K ion signal of dead 1 mer detected by the liquid-ELDI is varied with the amount of the acid.

FIG. 12 shows the configuration of the monitoring system.

FIG. 13 is a schematic diagram of inside of the closed chamber including a liquid-ELDI ionization source system in connection with Q-TOF-MS.

FIG. 14 is a schematic diagram of the outside of the closed chamber including a liquid-ELDI ionization source system in connection with Q-TOF-MS.

FIG. 15 is an enlarged layout of internal pipelines connected to the reactor within the closed chamber.

FIG. 16 shows certain parameters of the mass spectrometer for 5+1 mer synthesis.

FIG. 17 shows the average mass spectrograms obtained during different 5+1 mer oligonucleotide synthesis by using one embodiment of the monitoring system of the present invention: (a) the background mass spectrogram only with ESI (b) before the beginning of the reaction (c-h) 0 min, 5 min, 10 min, 15 min, 20 min and 25 min after the beginning of the reaction, respectively.

FIG. 18 shows the extracted ion chromatogram (EIC) of analytes obtained from the 5+1 mer oligonucleotides synthesis by using one embodiment of the monitoring system of the present invention: m/z 2482 (5 mer) and m/z 3242 (6 mer).

### DETAILED DESCRIPTION OF THE PRESENTLY PREFERRED EMBODIMENTS

### The Monitoring System of the Present Invention

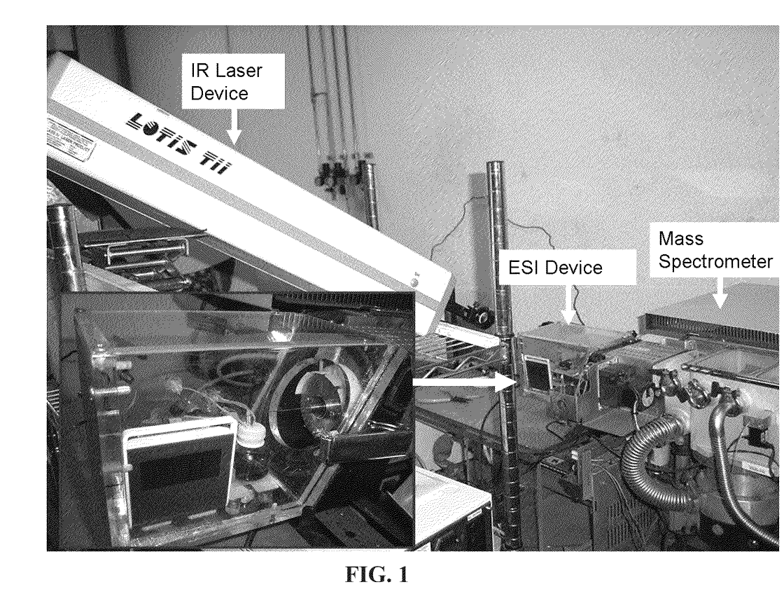

In order to ensure that the Liquid-ELDI analysis is completely undertaken under an anhydrous environment to facilitate the detection of the products of RNA synthesis, the present invention provides a monitoring system wherein the reactor and the ESI device are within a closed chamber (as shown in FIG. 1). The closed chamber provides a low-humidity environment, which cannot be achieved by the conventional open system. One embodiment of the present invention provides a system for real-time continuously monitoring oligonucleotide synthesis in solution phase, comprising an ion trap mass spectrometer, a pulsed infrared (IR) laser device, and a closed chamber, wherein the chamber contains an ESI device and a reactor (as shown in FIG. 5) and is capable of providing an anhydrous environment.

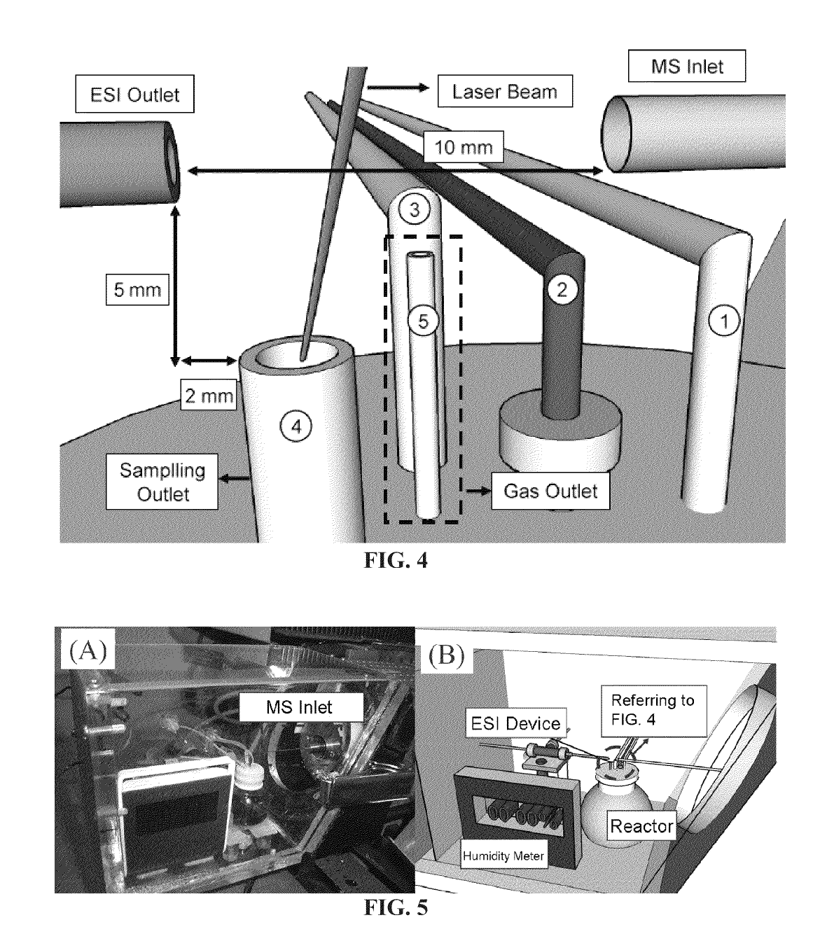

In one embodiment of the present invention, the layout of the closed chamber is illustrated in FIGS. 4 and 5. The reactor was filled with nitrogen to remove moistures in the atmosphere. Then a trace of the solution in the reactor was pushed into a stainless steel capillary (the sampling outlet) by nitrogen gas. After the solution is blown out of the capillary, it was desorbed by laser, and then the desorbed gaseous analyte molecules, including neutral oligonucleotides, were ionized within an electrospray (ESI) plume to generate ESI-like analyte ions. The produced analyte ions were detected by the mass spectrometer connected with the chamber.

### Internal Layout of the Closed Chamber:

Because the intensity and variation of analyte ion signals would be affected by the relative positions of the outlet of ESI capillary (ESI outlet), the sampling outlet beamed by laser, and the inlet of mass spectrometer (MS inlet), standard solutions were analyzed to determine the optimum conditions before the samples from the reaction mixture of the RNA synthesis were analyzed. In one embodiment of the present invention, the distances between each pipeline are illustrated in FIG. 4, which represents the optimum relative positions of the aforementioned devices. And this layout is generated based on the analysis results of the standard solutions.

### System Humidity Control:

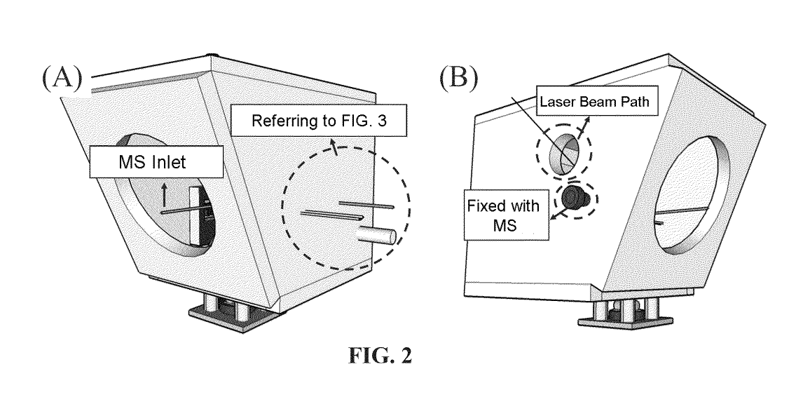

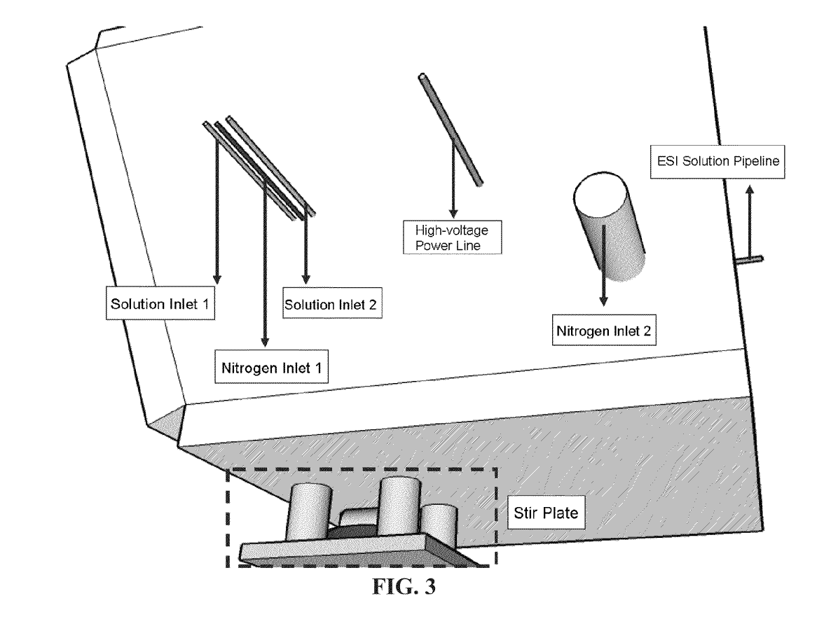

Because the process for oligonucleotide synthesis must be conducted in low-humidity environment at all times, we enclosed the entire inlet of the mass spectrometer (MS inlet) in the closed chamber. In order to avoid outside air and moisture entering the reactor through the space beneath the MS inlet, we installed a nitrogen inlet 2 on the side of the chamber as shown in FIG. 3, wherein nitrogen gas flowed into the nitrogen inlet 2 from an internal pipeline of the ion trap mass spectrometer. In addition, before adding reactants into the reactor containing the solutions, nitrogen gas was introduced into the reactor through the nitrogen inlet 1 as shown in FIG. 3 to flush out the moisture from the reactor. The moisture of the chamber was also brought out by the nitrogen gas flow via the space beneath the MS inlet. This introducing nitrogen gas step can prevent the inflow of outside moistures and effectively lower the humidity inside the reactor so the oligonucleotide synthesis can be conducted successfully.

### Sampling of the Reaction Mixture:

In order to constantly push the reaction mixture to flow out of the sampling outlet of the reactor (as shown in FIG. 4), where the sample is irradiated by laser to produce the analyte droplets, an nitrogen flow is introduced into the reactor via the nitrogen inlet 1 to increase the inside pressure, so as to propel the reaction mixture out of the sampling outlet (as shown in FIG. 4). In addition, the nitrogen gas pressure on the reaction mixture needs to be adjusted during the analysis process, so that the speed of reaction mixture flowing out of the reactor can be controlled. Excessive nitrogen gas pressure would cause an overflow of reaction mixture, resulting in variable signals for analysis, but if nitrogen gas pressure is too low, the reaction mixture cannot be propelled out of the reactor.

### Example

### Continuous Monitoring of 3+1 mer Oligonucleotide Synthesis:

In order to prevent outside moisture from interfering with the oligonucleotide synthesis, prior to the start of synthesis, nitrogen gas was fed into the closed chamber and the reactor containing 300 mg carbon powders via the nitrogen inlet 2 and the nitrogen inlet 1 respectively (as shown in FIG. 3). The nitrogen gas feeding lasted about 20 minutes and then the nitrogen inlet 1 was shut off after the humidity in the closed chamber was reduced to 0%. Next, the pre-prepared RNA trimers, RNA monomers and the activators were injected into the reactor through the solution inlet 1 and the solution inlet 2. As the reaction was taking place, the nitrogen inlet 1 was opened to propel the reaction mixture out of the sampling outlet as shown in FIG. 4. The solution flowing out of the sampling outlet was desorbed by laser, ionized by the ESI device and then subjected to the ion trap mass spectrometer connected thereafter for the continuous monitoring.

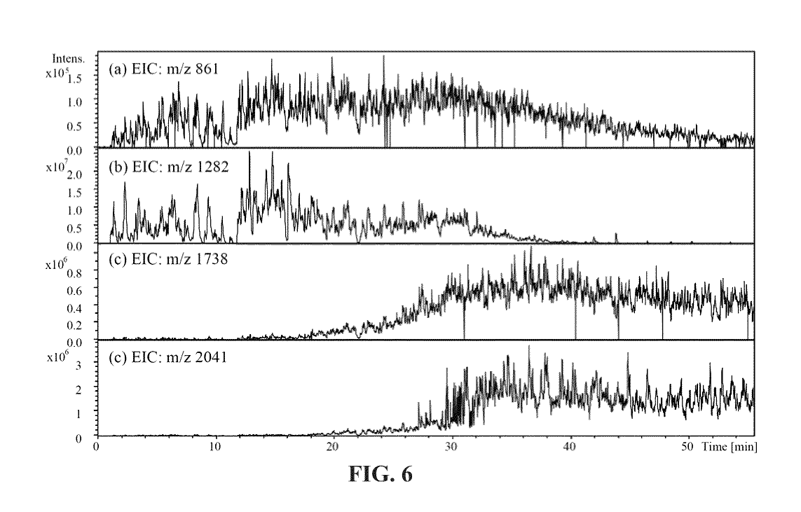

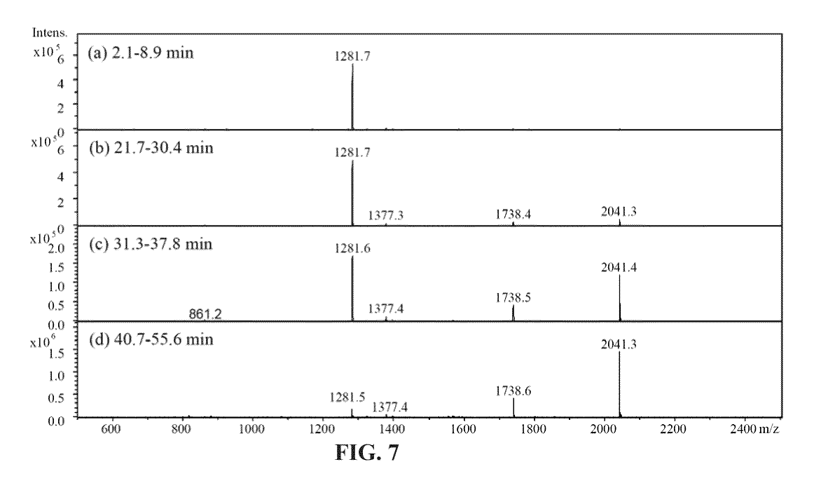

In the continuous monitoring of 3+1 mer oligonucleotide synthesis, the analyte ions of reactants, [1mer+H]⁺ (m/z 861) and [3mer+H]⁺ (m/z 1282), as well as the analyte ions of products, [(4mer-DMT)+H]⁺ (m/z 1738) and [4mer+H]⁺ (m/z 2041), are subjected to extract ion chromatogram (EIC). As shown in FIG. 6, after 9 minutes from adding the reactant, 1mer (RNA monomers), into the solution containing 3mer (RNA trimers) and activators, the analyte ion signals of reactants, 1mer (m/z 861) and 3mer (m/z 1282), became weaker and weaker over the reaction time. Conversely, the analyte ion signals of products, 4mer (m/z 2042) and 4 mer-DMT (m/z 1738), were getting stronger over the reaction time. FIG. 7 shows mass spectrograms of analytes at different reaction intervals. FIG. 7(a) shows the average MS signals obtained during the interval of 2.1-8.9 minutes. Because this is the initial stage of the synthesis, the spectrogram mainly shows the analyte ion signals of the reactant (3mer). After adding 1mer and activators over a period of time, the analyte ion signals of the product (4mer) and de-DMT 4mer fragment became stronger and stronger (as shown in FIGS. 7 (b) and (c)), while the signals of the reactants (3mer and 1mer) became weaker gradually. FIG. 7 (d) shows the mass spectrogram obtained from the final stage of the synthesis, displaying mainly the analyte ion signals of the product (4mer) and de-DMT 4mer fragment.

The results of continuous online monitoring of 3+1 mer oligonucleotide synthesis show that the closed liquid ELDI device in the present invention can effectively prevent outside moisture from interfering with the synthesis reaction. The EIC and mass spectrograms obtained by using the monitoring system of the present invention can help chemists or engineers to understand the kinetics within the reaction mixture of the oligonucleotide synthesis. The present invention can also be applied to monitor even much higher molecular weight polynucleotide synthesis in solution phase.

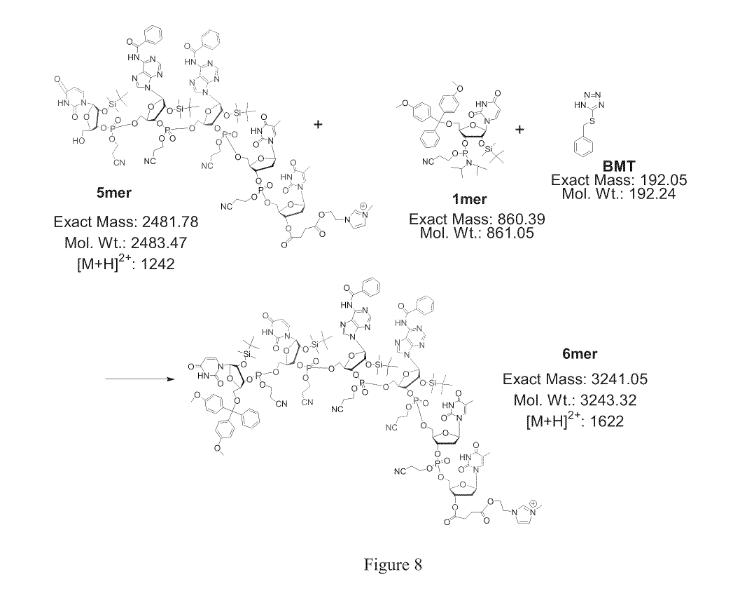

In another embodiment of the present invention, the real-time and continuous monitoring for the synthesis of RNA hexamer (5+1 mer) as shown in FIG. 8 is provided. However, when the system used for monitoring the synthesis of RNA tetramer is applied to monitor the synthesis of hexamer, two problems are observed.

### (1) Reaction Environment:

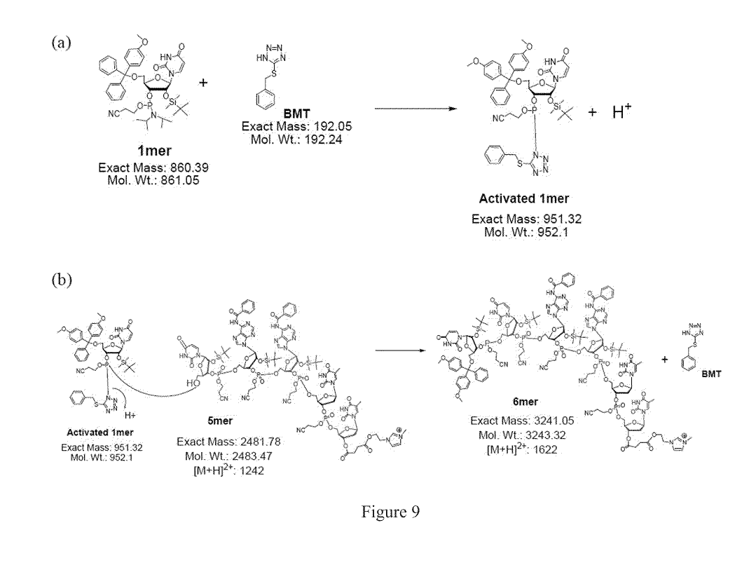

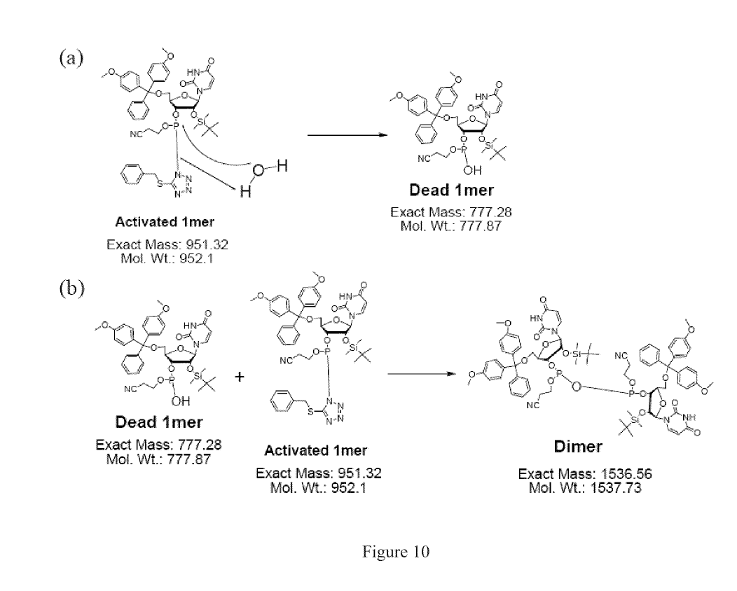

FIG. 9 shows 5+1 mer RNA synthesis mechanism. The reaction of 1 mer and benzylmercaptotetrazole (BMT) produces an activated 1mer, which in turn is reacted with 5 mer to produce the 6 mer product. When there is moisture in the reaction solution or the environment, water will react with the activated 1 mer to produce dead 1 mer (See FIG. 10). However, dead 1 mer will not react with 5 mer to produce 6 mer, so a lot of 5 mer will be unreacted. In addition, since the structure of 5 mer is more distorted than that of 3 mer, hydroxyl on 5 mer is more hindered, so the reaction efficiency will be decreased. Therefore, the amount of 1 mer added to 5+1 mer synthesis is more than that added to 3+1 mer synthesis in order to increase the reaction efficiency. To remove moisture from the system and the electrospray solvent is very important since the ion suppression caused by dead 1 mer will become more serious when moisture exists in the system.

### (2) Mass Spectrometer:

The detection limit of the ion trap mass spectrometer is m/z=3000 Da, so the ion trap mass spectrometer can detect the signals related to the monovalent- or divalent ion of 5 mer (MW.=2481) and the divalent ion of 6 mer (MW.=3241). However, many signals related to dead 1 mer, such as [dead 1 mer+K]⁺, [dimer-dead 1 mer+Na]⁺, will seriously suppress the ion signal of the reactant 5 mer and the product 6 mer such that the relative variation between 5 mer and 6 mer cannot be observed. If the monovalent ion signal of 6 mer can be obtained directly, the ion suppression effect can be avoided. The mass-to-charge ratio of the final product 6 mer monovalent ion (m/z>3000 Da) is more than the maximum detectable mass-to-charge ratio, so the ion trap mass spectrometer cannot detect the signal of 6 mer monovalent ion.

In view of the above two points, when monitoring 5+1 mer RNA synthesis, Quadrupole Time-of-Flight Mass Spectrometry (Q-TOF-MS) is preferably used as the mass analyzer instead of the ion trap mass. The monovalent ion signal of 5 mer and 6 mer can be obtained and the ion suppression caused by the lower molecular weight substances is decreased because of the resolution for the high molecular weight substance of Q-TOF-MS. Meanwhile, the voltage of Quadrupole is under pure radio-frequency (RF) such that all ions regardless of the value of m/z can pass the Quadrupole, enter the TOF and be detected. Therefore, the monovalent ion signal of 6 mer can be obtained. Another advantage of Q-TOF-MS is that the intensity of the ion signal related to dead 1 mer can be decreased by varying the above parameters so the suppression for the signals of 5 mer and 6 mer is also decreased.

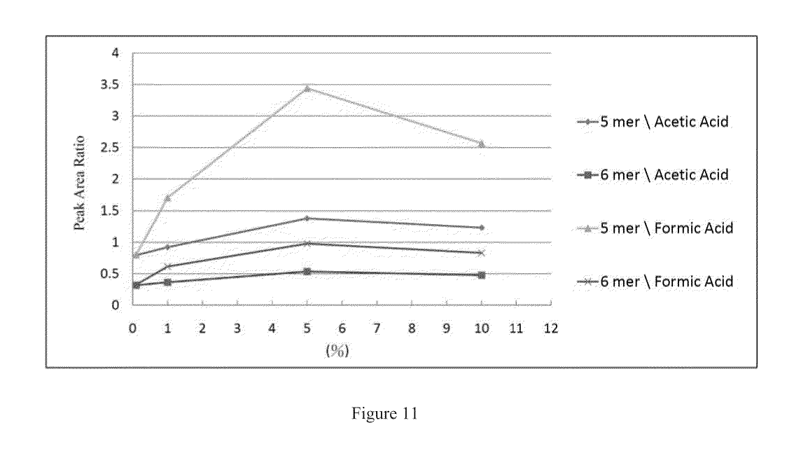

Regarding the reaction environment, in addition to designing a reactor that is isolated from moisture, the composition of the electrospray solution is also changed to methanol:formic acid=99:1 (v/v) from methanol: water: acetic acid=49.95:49.95:0.1 (v/v/v) for 3+1 mer synthesis. The content of the acid in the electrospray solution also affect the ion signal of each analytes in the reaction solution. Different electrospray solutions which comprise different amounts of acetic acid or formic acid are applied to the closed liquid-ELD ionization source system to detect the solution resulted from the 5+1 mer synthesis. The result is shown in FIG. 11, wherein the X axis is the amount of the acid (acetic acid or formic acid) and Y axis is the corresponding peak area ratio. Theoretically, when the same acid is used, the higher the ratio of the acid, the better the ionization of the analytes. Therefore, the ion signal with the better intensity will be obtained. In the solution system of the present invention, the more the amount of the acid, the higher the intensity of the ion signal of 5 mer and 6 mer. However, the intensity of the ion signal related to dead 1 mer is not positively correlated with the amount of the acid. Therefore, the ion suppression effect caused by the dead 1 mer can be decreased by increasing the ion signal of 5 mer and 6 mer. In addition, it is observed that the ionization of the RNA species is better for formic acid than acetic acid since the acidity of formic acid is higher. Therefore, the intensity of the ion signal of 5 mer and 6 mer for formic acid is much higher than acetic acid. It is also observed that the optimum signal can be obtained when the amount of formic acid is 5%. When the concentration of the acid (10%) is higher, the ion signal of 5 mer and 6 mer are decreased. That may be because as the huge amount of the acid increases the ionization of the species, more related ion signals appear such that the signal of monovalent ion is decreased. The mass spectrometer is mainly composed of metallic materials. The amount of the acid used in the electrospray solution cannot be too high since the acid at high concentration will harm the spectrometer. Therefore, when monitoring the reaction by liquid-ELDI techniques of one embodiment of the present invention, 1% of formic acid is used.

### Equipment:

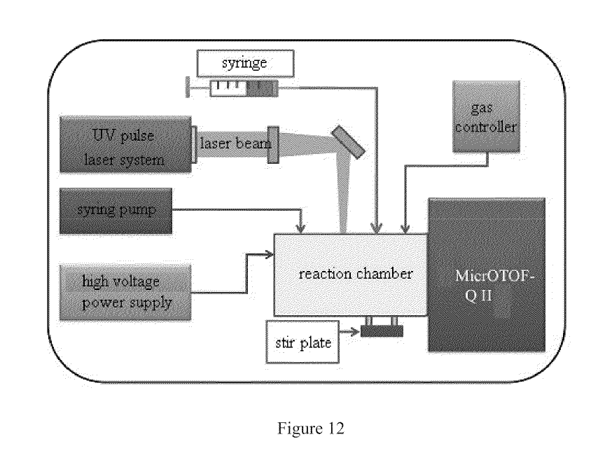

The configuration of the monitoring system is shown on FIG. 12.

1. Mass spectrometer: Produced by Bruker Dalton, Trade name: microTOFQ II, Quadrupole Time-of-Flight Mass Spectrometry (Q-TOF-MS)

2. Pulse laser system: Produced by Continuum company, Trade name: MINILITE I. Laser light source is focused by single convex lens (diameter: 24.5 mm, focal length: 150 mm). In addition to the convex lens, a reflection mirror and a light window are also used. The frequency and the intensity of laser are respectively 10 Hz and 400 μJ.

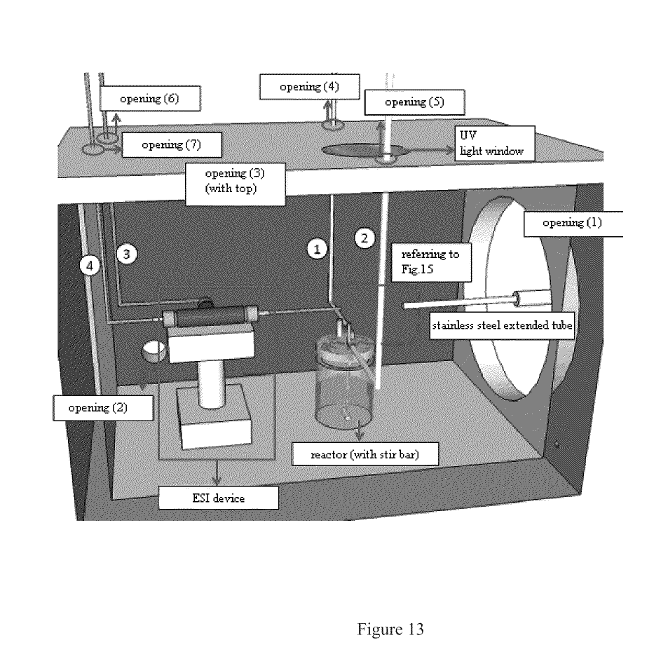

3. Reaction Chamber: The inside of the chamber is shown in FIG. 13.

3-1. electrospray ionization source system

a. Fused Silica Capillary
b. Syring Pump
c. High Voltage Power Supply
d. The entrance of the Mass-stainless steel extended tube

3-2. Reactor:

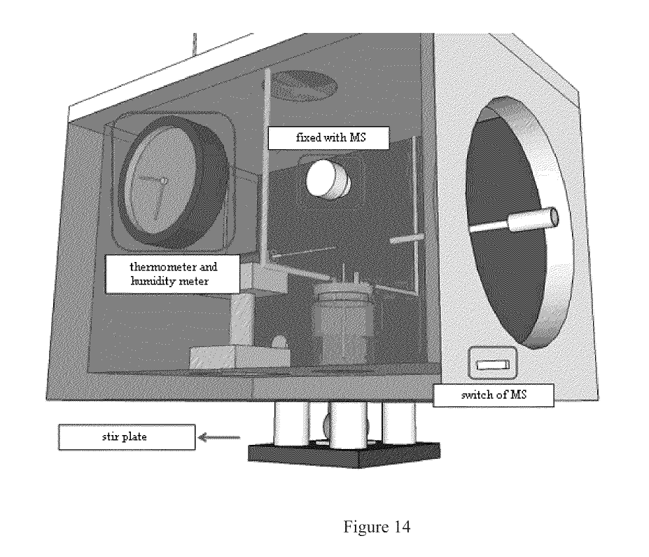

The outside of the reactor body is shown on FIG. 14.
Stir Plate (under the reactor, as shown on FIG. 14):

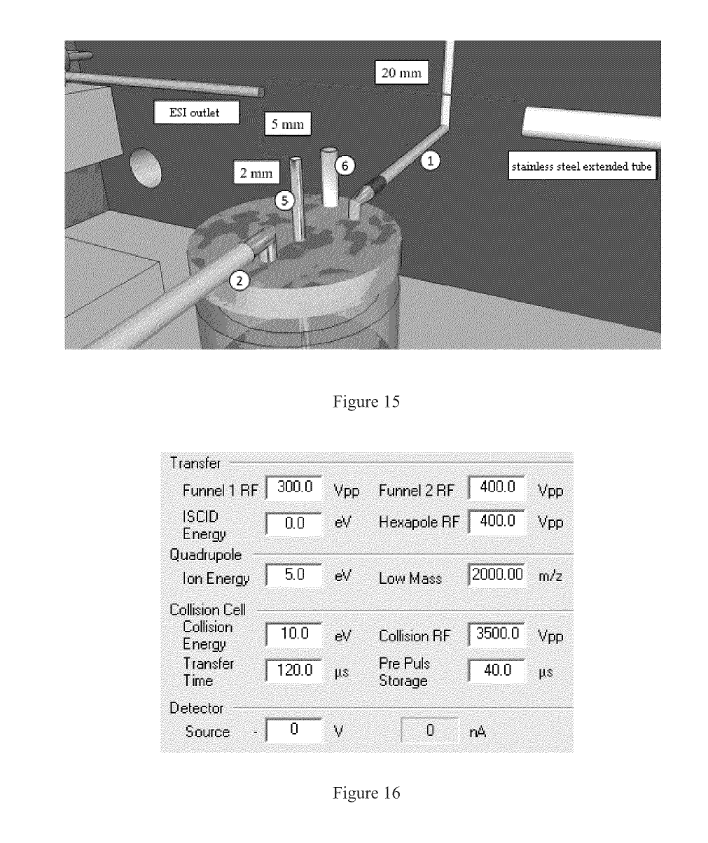

3-3. The reaction bottle is shown in FIG. 15:

3-4. pipelines configuration:

a. nitrogen inlet (1 on FIG. 15):
b. reaction solution inlet (2 on FIG. 15):
c. high-voltage power line and fused silica capillary (3 and 4 on FIG. 13):
d. stainless tube (5 and 6 on FIG. 15):

### The Preparation of the Reactants

1. A pentamer solution was made by adding 0.5 mL of anhydrous acetonitrile and 0.5 mL of anhydrous Dimethylformamide into a glass bottle containing 25 mg of RNA pentamer

2. A monomer solution was prepared by adding 3 mL of anhydrous acetonitrile to 100 mg of monomer.

### Continuous Monitoring of 5+1 mer Oligonucleotide Synthesis:

The parameters of the mass spectrometer are set as shown on FIG. 16. The electrospray ionization source system and the reaction bottle with a stir bar are put into the reactor. The magnetic stirrer is placed under the reactor. The flow rate of the electrospray solution is set at 0.20 mL/hour. Finally, the high voltage power supply is turned on to perform the electrospray. Before beginning of the reaction, nitrogen gas is fed into the reaction bottle via 1 until the system is dried thoroughly. 1 mL of 5 mer solution and 1 mL of BMT solution were injected into the reaction bottle via 2. 0.5 mL of anhydrous ACN is added such that the liquid level of the reaction solution is higher than the bottom of 5. The reaction solution was stirred. The nitrogen inlet 1 was opened to propel the reaction mixture out of the sampling outlet 5. The solution flowing out of the sampling outlet was desorbed by pulse laser. After the stable ion signal was obtained, 3 mL of 1 mer solution was injected to the reaction bottle via 2. The data of the mass spectrogram was collected every 5 minutes.

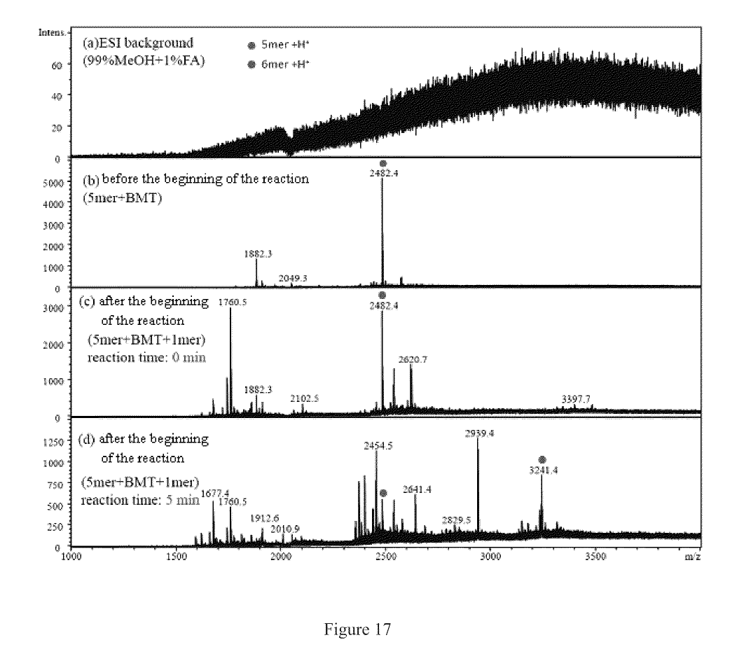

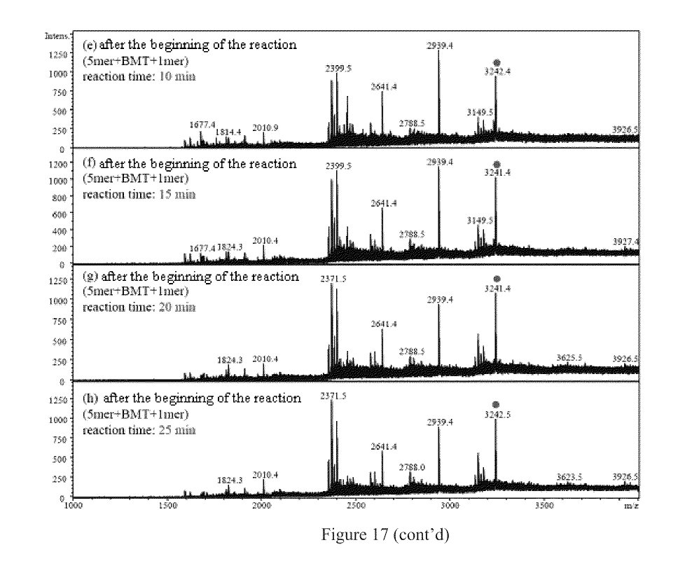

The result is as shown on FIG. 17. FIG. 17(a) shows the background mass spectrogram obtained before the addition of the reactants. FIG. 17(b) shows the mass spectrogram obtained after the addition of 5 mer and BMT solution. The monovalent ion signal of 5 mer can be observed. The ion signal m/z 1882.3 may be the fragment of mer produced during the electrospray. FIG. 17 (c-h shows the mass spectrogram obtained after the addition of 1 mer solution. It can be observed that the ion signal of 6 mer increased over time and the ion signal of 5 mer decreased gradually.

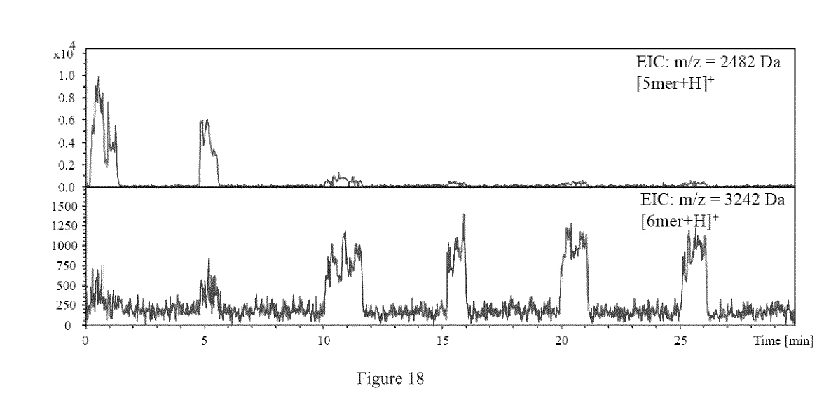

The monovalent ion signals of 5 mer and 6 mer are subjected to extracted ion chromatogram (EIC), as shown in FIG. 18. 5 minutes after the beginning of the reaction, the analyte ion of 5 mer became weaker over the reaction time. Conversely, the analyte ion signal of product, timer, was getting stronger over the reaction time. By 15 minutes after the beginning of the reaction, almost all 5 mer was converted to 6 mer.

The UV pulse laser of one embodiment the present invention is used to desorb the reaction solution. Usually, when the RNA species are exposed to UV laser, the RNA species may decompose. However, the clear ion signal of the RNA reactant or product is still observed in the present invention. Therefore, desorption effect seen in the laser desorption system of the liquid-ELDI is far more than that from a UV laser.

The closed liquid-ELDI system which is in connection with is dried more thoroughly but the suppression effect caused by dead 1 mer is also decreased by setting the parameter of Q-TOF-MS. The composition of electrospray solution also improve the ion signals of the reactant 5 mer and the product 6 mer and decreases the production of dead 1 mer. In addition, the dynamics information relating to the reactant and the product and the variation of the amount of the reactant and the product can be observed by the mass spectrogram and the extracted ion chromatogram. Therefore, the closed liquid-ELDI ionization source system can monitor the RNA synthesis without the complicated sample pretreatment.

The coupling step is a critical step in oligonucleotide synthesis. The coupling efficiency and completion of reaction will affect final product's purity and yield. In solution phase oligonucleotide synthesis, due to the large structure and diastereomer effect, there are no suitable analytical method to monitor the coupling reaction. The present invention was developed to continuously monitor the coupling reaction. By observing the decreasing of the starting material signals and increasing of the product signals, it can judge the coupling efficiency and make sure the reaction is completed. Also, the detection equipments combined with the reactor having the oxygen and moisture eliminated condition can provide the ideal environment for oligonucleotide synthesis and the monitoring thereof.

### REFERENCES

Anal. Chem. 2008, 80, 4845-4852
Anal. Chem. 2008, 80, 7699-7705
US20080308722
US20080116366
US20080006770
US20070176113

We claim:

1. A method for monitoring the synthesis of oligonucleotides, comprising adding the reactants and conducting the synthesis in solution in a reaction container having a first plurality of tubes, which reaction container is placed in a substantially moisture free chamber along with an electrospray-assisted laser desorption ionization (ELDI) device, wherein the chamber comprises a wall which has at least one portion that is transparent to a laser beam and a second plurality of tubes that connects the inside of the chamber to the outside, such that as the synthesis is being performed
a) a sample of the solution is moved through at least one of the first plurality of tubes out of the reaction container through a sampling outlet;
b) at least a portion of the sample that is outside the reaction container is desorbed by the laser beam into a gaseous sample comprising neutral oligonucleotides;
c) at least a portion of the neutral oligonucleotides is ionized by the ELDI device having an electrospray outlet; and
d) the ionized oligonucleotides are then transported out of the chamber through at least one of the second plurality of tubes for detection by a mass spectrometer.

2. The method according to claim 1 wherein the oligonucleotides that are synthesized are RNA tetramers or RNA hexamers.

3. The method according to claim 1 wherein dried nitrogen gas is flowed through the reaction container before adding the reactants of the synthesis.

4. The method according to claim 1 wherein the end of at least one of the first plurality of tubes is positioned in the solution to transport the sample through the sampling outlet.

5. The method according to claim 1 wherein the sample is moved through the sampling outlet by increasing the pressure within the reaction container above the pressure of the chamber.

6. The method according to claim 1 wherein the laser beam is produced by a UV pulse laser.

7. The method according to claim 1 wherein the end of the electrospray outlet is about 2 mm from the sampling outlet.

8. The method according to claim 1 wherein the end of the electrospray outlet is about 10 mm from the end of at least one of the second plurality of tubes for detection by a mass spectrometer.

9. The method according to claim 1 wherein the electrospray solution comprises a mixture of methanol, water and acetic acid or a mixture of methanol and formic acid.

10. The method according to claim 1 wherein the mass spectrometer is an ion trap mass spectrometer or a Quadrupole Time-of-Flight Mass Spectrometer.

11. A system for monitoring oligonucleotides synthesis in solution, comprising a reaction container containing the solution and having a first plurality of tubes, which reaction container is placed in a substantially moisture free chamber along with an electrospray-assisted laser desorption ionization (ELDI) device, a laser and a mass spectrometer that is placed outside the chamber;
wherein the chamber comprises a wall which has at least one portion that is transparent to a laser beam and a second plurality of tubes that connects the inside of the chamber to the outside, and wherein
e) the first plurality of tubes connects the inside of the reaction container to the outside of the reaction container to deliver a sample of the solution out of the reaction container through a sampling outlet;
f) the laser is capable of impinging a laser beam into the chamber so as to desorb at least a portion of the sample that is outside the reaction chamber into a gaseous sample comprising neutral oligonucleotides;
g) the ELDI device is capable ionizing at least a portion of the neutral oligonucleotides; and
h) at least one of the second plurality of tubes is capable of transporting the ionized oligonucleotides out of the chamber for detection by the mass spectrometer.

12. The system according to claim 11 wherein at least one of a humidity sensor and a temperature sensor is placed inside the chamber.

13. The system according to claim 11 wherein the laser is an UV pulse laser.

14. The system according to claim 11 wherein the mass spectrometer is an ion trap mass spectrometer or a Quadrupole Time-of-Flight Mass Spectrometer.
